(19) United States
(12) Patent Application Publication
(10) Pub. No.: US 2025/0049641 A1
(43) Pub. Date: Feb. 13, 2025

(54) PILL DISPENSING SYSTEM AND METHOD

(71) Applicant: Donald T. Sanders, Indianapolis, IN (US)

(72) Inventor: Donald T. Sanders, Indianapolis, IN (US)

(21) Appl. No.: 18/931,384

(22) Filed: Oct. 30, 2024

(51) Int. Cl. A61J 7/00; A61B 5/1172; A61J 1/03; G16H 20/13; G16H 80/00

(52) U.S. Cl. CPC A61J 7/0076; A61B 5/1172; G16H 20/13; G16H 80/00; A61J 1/03

(57) ABSTRACT

A pill dispensing system is provided. The system includes a pill container having a base that houses a dispensing mechanism for dispensing a pill, a lid having a fingerprint reader, circuitry to read the fingerprint information of a patient, and drive a rotary motor. The pill container has a sleeve with sidewalls, a top opening, and a bottom opening. The sleeve, the lid, and the base engage with each other to retain pills inside the sleeve. The engagement is such that it creates child-resistant packaging to reduce the risk of children ingesting pills. The system includes a fingerprint information transfer medium equipped with a fingerprint scanner/reader. The fingerprint transfer medium records the patient's fingerprint and electronically transfers the recorded patient's fingerprint information to the pill container to link the patient's fingerprint to the pill container to allow the patient to authenticate and dispense the pills out of the pill container.

### TECHNICAL FIELD

The invention relates to medication dispensing containers in general. More particularly, the invention relates to a pill dispensing system and method that dispenses intended doses of pills for a patient in response to validating pre-recorded thumbprints or fingerprints of the patient according to an embodiment. Further, the proposed pill dispensing system and method is capable of dispensing pills without requiring thumbprints or fingerprint authentication according to another embodiment. Additionally, the proposed pill dispensing system and method is capable of tracking and assessing at least the status of pills held within the pill dispensing container, and timely intake of the pills by patients. The system can transmit alerts to the patients and pharmacy.

### BACKGROUND

Medicines to be taken orally are often supplied in tablet/capsule form. Medicinal tablets and capsules are often called pills. Medication containers have been used to assist individuals in storing medications and in maintaining a medication schedule.

The usage of medication in the form of pills to regain and maintain physical and mental health, and the selection, application, and potency of prescription medications have increased with the advancement of medical science. Likewise, the self-taking of prescribed medications by individuals has also increased over the years. The increase in self-administration of medication/pills without proper supervision from health personnel or pharmacists has led to an adverse effect on a patient who may either access the wrong medication (especially elderly people) or consume the incorrect dosage of medication due to no proper restriction on dispense of the pills stored in the container. Also, due to a lack of proper indicators or alerts to the patients, the patients often tend to forget about the intake of the medication/pills that they are supposed to take. Further, the available medication containers used for holding the pills lack the proper means to restrict the access to medications held therein, especially access to infants, small children, and other unintended users such as a repair person or other service providers visiting a patient.

Several incidents have been documented in the past where the patient has either taken an overdose of the medication or identified the wrong pill bottle, and where the pill bottles have been accessed by infants and children without the knowledge of the patient. These unfortunate actions can lead to various adverse effects on the user's life, as well as those associated with the user, including health risks, and risks associated with the infant/children or unintended users accessing the pill bottles or containers.

Housed within the pill bottles/containers, traditionally there are no secure means of protecting the pills apart from a child-proof locking mechanism. These child-proof closures are designed to create a sense of frustration in infants and small children who try to access and open the pill bottles/containers. Commonly utilized designs of such child-proof closures include caps/lids that are required to be aligned in a specific rotational orientation to permit opening, caps that require downward pressure on the top of the cap to engage an inner cap for the necessary unscrewing rotations or to release the cap from a locking device. Although these known child-proof closure designs are reasonably effective for their primary intended purposes, they cause difficulty for many adults to open to intake their medications, particularly elderly persons with poor eyesight or physical problems with their hands.

There is a need to provide security in connection with access to and dispersing of pills. There are also circumstances where it is desirable to know who and when an individual has had access to the prescribed pill container in addition to securing the pills from infants, small children, and other unintended users visiting the residence of the patient. Further, traditionally known pills dispensing containers do not impose any restriction on the number of pills to be dispensed under a single dose for a patient which may impose the risk of consuming an overdose of the pills.

Also, traditionally available pill dispensing containers are unable to alert the patients and the pharmacies about appropriate doses of the pills one needs to intake, about the time when the patient needs to take his/her pills, about the pills remaining in the pills dispensing containers, alerting pharmacy and/or health professional about if the patient is taking his/her pills on time or not, and so on.

Some smart pill dispensers for home use have been developed in the past as well. For example, US2009223994 discloses a thumb/fingerprint-activated pill dispenser that uses a technique of fingerprint recognition for locking and unlocking a cap with the help of a computer chip. US2019365607 discloses a dispensing mechanism whose functional parameters such as dosage to be dispensed, time for dispensing dosage, access to the authorized user, etc. are predefined by the pharmacists. U.S. Pat. No. 10,872,482 discloses a personalized lid for prescription bottles having a biometric sensor for unlocking the bottle cap using a latch. CN111096898 discloses a smart drug box and control method thereof having a biometric sensor preferably a fingerprint sensor for locking and unlocking the lid.

Although to some extent these prior existing solutions solve the problem of unauthorized access to pill containers holding the pills, they aren't designed to meet diversified demand from patients and pharmacies.

### BRIEF SUMMARY

According to an embodiment, provided is a pill dispensing system and method that activates a dispensing mechanism housed within a pill dispensing container to dispense the intended doses of pills for a patient in response to validating one or more pre-recorded thumbprints or fingerprints of the patient.

According to another embodiment, provided is a pill dispensing system and method that involves the use of a pill dispensing container, a communication device associated with a patient, and a pharmacist having a computer program product configured therein to provide alerts to the patients and the pharmacists. The alerts may be related to pill intake by the patient, remaining doses of pills in the pill dispensing container, and so on.

It is an object of the present invention to provide a pill dispensing container adapted for holding the prescribed pills prefilled by a pharmacist, wherein the pill dispensing container is linked to a patient's fingerprint information at the time the patient purchases prescribed pills from the pharmacist. The patient is required to authenticate himself/herself with fingerprint information linked to the pill dispensing container before dispensing the pills.

It is an object of the present invention to provide a novel pill dispensing mechanism housed within the pill dispensing container that includes a rotating disc that is turned a full rotation by a rotary motor to dispense a pill out of the pill container.

It is an object of the present invention to provide a pill container/dispenser that can be used to hold and dispense pills of different sizes by the rotating disc that includes a plurality of dispensing slots of different sizes in line with the standard pill sizes available in the market.

It is another object of the present invention to provide a fingerprint information transfer station configured in the form of a stationary product or a portable handheld device equipped with a fingerprint scanner/reader, wherein the fingerprint transfer station or the portable handheld device is capable of recording the patient's fingerprint information and electronically transferring the same to a server and linking the information to the pill container or any specific portion thereof before the delivery of the pill dispensing container to the patient.

It is an object of the present invention to provide a pill container/a pill dispenser that facilitates tracking the number of pills dispensed in total, the number of pills dispensed in a specific day, the number of access to the pill container done by the patient, number of pills remaining and so on.

Various other advantages and features of the present invention are described herein with specificity so as to make the present invention understandable to one of ordinary skill in the art, both with respect to how to practice the present invention and how to make the present invention.

### BRIEF DESCRIPTION OF THE DRAWINGS

Elements in the figures have not necessarily been drawn to scale to enhance their clarity and improve understanding of these various elements and embodiments of the invention. Furthermore, elements that are known to be common and well understood to those in the industry are not depicted in order to provide a clear view of the various embodiments of the invention. The novel features which are believed to be characteristic of the present invention, as to its structure, organization, use, and method of operation, together with further objectives and advantages thereof, will be better understood from the following drawings in which a presently preferred embodiment of the invention will now be illustrated by way of various examples. It is expressly understood, however, that the drawings are for the purpose of illustration and description only and are not intended as a definition of the limits of the invention. Embodiments of this invention will now be described by way of example in association with the accompanying drawings in which:

FIG. 1 is a schematic diagram showing a fingerprint information transfer station configured at a pharmacy for transferring or linking fingerprint information to a pill container, according to an embodiment of the present invention.

FIG. 2 shows a handheld fingerprint information transfer device usable at a pharmacy for transferring or linking fingerprint information to a pill container, according to another embodiment of the present invention.

FIG. 3 shows connectivity between the handheld fingerprint information transfer device of FIG. 2 with a lid of the pill container for transferring or linking fingerprint information to a pill container.

FIGS. 4-5 shows perspective views of the pill container according to an embodiment of the present invention.

FIG. 6 shows an exploded view of the pill container/dispenser of FIG. 4.

FIG. 7 is a cross-sectional view of the pill container/dispenser of FIG. 4 taken along A-A.

FIG. 8 shows a simplified pill container/dispenser of FIG. 4 with a lid and sleeve removed.

FIG. 9 shows another exploded view of the pill container/dispenser of FIG. 4.

FIG. 10 shows a pill container according to another embodiment of the present invention.

FIG. 11 shows a schematic diagram involving the use of the pill container of FIG. 10 and an app installed on a user's device and a pharmacist's device at a pharmacy, according to an embodiment.

### DETAILED DESCRIPTION

As used in the specification and claims, the singular forms “a”, “an” and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention. There may be additional components described in the foregoing application that are not depicted in one of the described drawings. In the event such a component is described, but not depicted in a drawing, the absence of such a drawing should not be considered as an omission of such design from the specification.

Before describing the present invention in detail, it should be observed that the present invention utilizes a combination of components, which constitutes a pill dispensing system and a method for dispensing pills out of a pill dispenser after authenticating a patient. Accordingly, the components have been represented, showing only specific details that are pertinent for understanding the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising”, “having”, “containing”, and “including”, and other forms thereof, are intended to be equivalent in meaning and be open-ended in that an item or items following any one of these words is not meant to be an exhaustive listing of such item or items or meant to be limited to only the listed item or items. Unless stated otherwise, terms such as “first” and “second” are used to arbitrarily distinguish between the elements or entities. Thus, these terms are not necessarily intended to indicate temporal or other prioritization of such elements or priorities. In the following description, the terms “pill container”, “pill dispenser”, “pill dispensing container”, and the like terms are all interchangeably used and referrer to a container that's meant to hold the pills desired by the patient, that may then is capable of dispensing the pills in predefined doses when desired by a patient. The terms “computer program product”, “software program product”, “program product”, “an app” and so on as used in this disclosure essentially mean a set of instructions stored in the memory of the device that when executed by a processing component of the electronic communication devices associated with the patient, pharmacist, etc is capable of generating an output on the display screen of the devices.

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments. Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example and simply illustrates certain example embodiments.

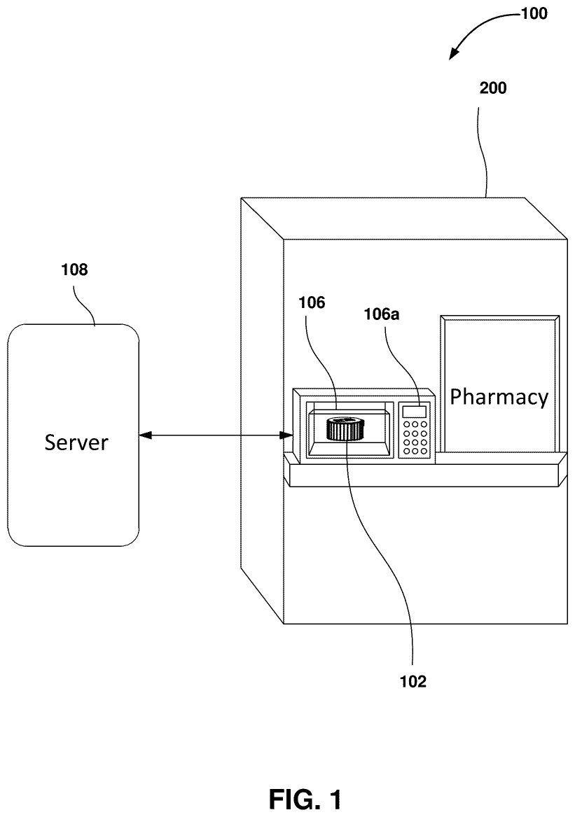

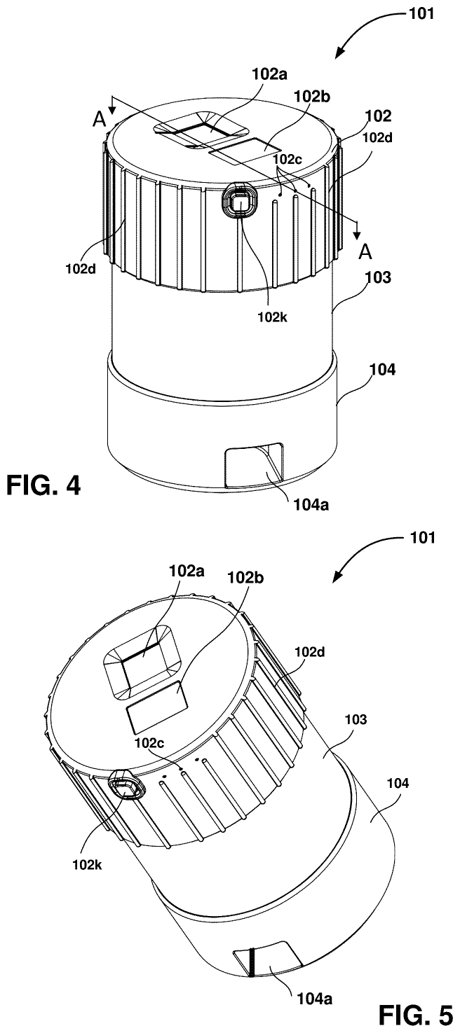

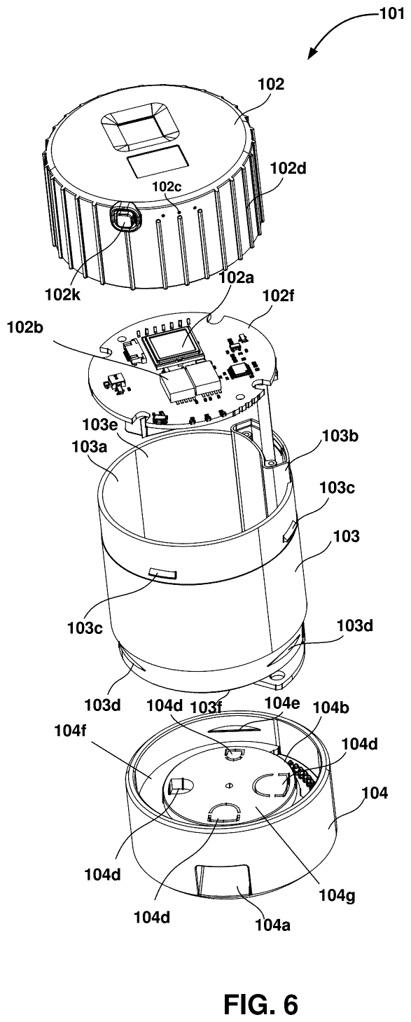

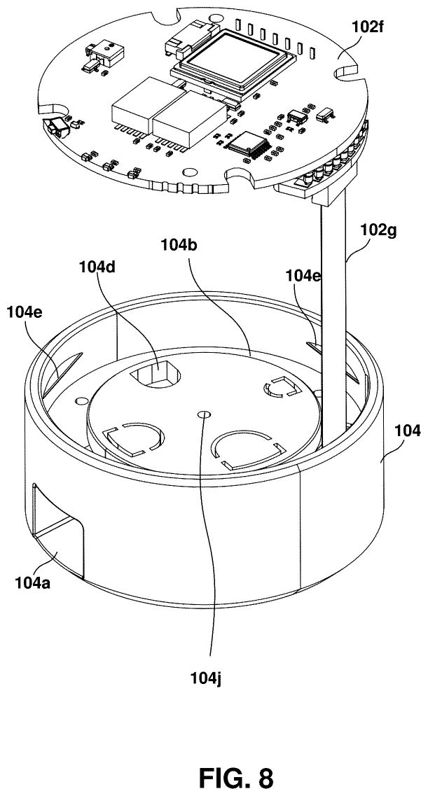

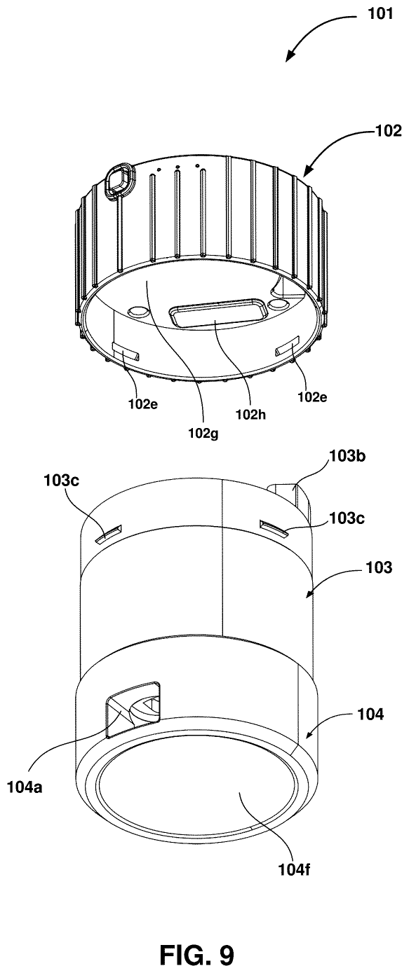

The pill dispensing system and associated method thereof according to various possible embodiments will now be described with reference to the accompanying drawings particularly FIGS. 1-11, which should be regarded as merely illustrative without restricting the scope and ambit of the present invention.

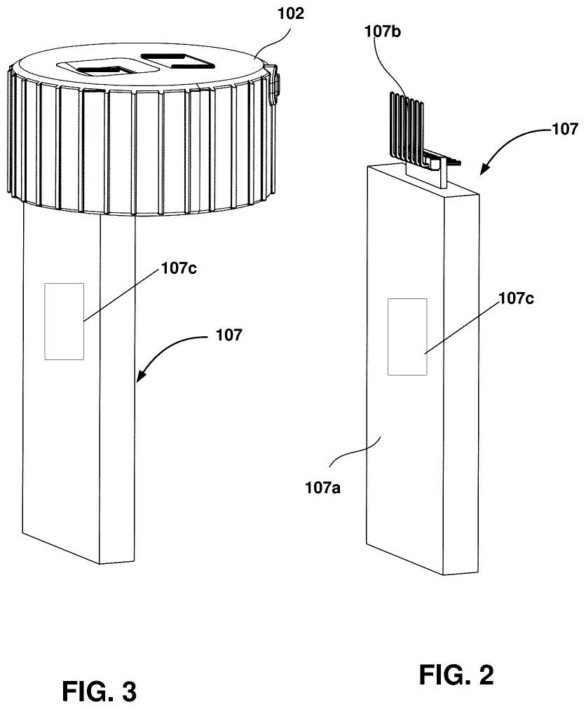

Referring to the accompanying figures, particularly FIG. 1 where a pill dispensing system 100 of the present invention is shown. The pill dispensing system 100 primarily shows a fingerprint information transfer station 106. The fingerprint information transfer station 106 is usually configured at pharmacy 200 at some appropriate location. In the context of the present invention, the pharmacy 200 refers to a resource or a place that makes prescribed medication available to patients with a valid prescription. Further in the context of the present disclosure, the term ‘patient’ refers to any user or purchaser of the prescribed medicines/pills from the pharmacy 102. The fingerprint information transfer station 106 may be configured in many different forms/designs that would allow a patient 202 visiting the pharmacy 200 to scan his fingerprints/thumbprints using a fingerprint scanner/reader 106a associated therewith. FIG. 1 shows the fingerprint information transfer station 106 configured in the pharmacy as a stationary object or box-like object. However, it is possible that the fingerprint information transfer station 106 to be configured in the form of a handheld device 107 as shown in FIG. 2. In the context of the present disclosure, the use of fingerprints/thumbprints is disclosed as a preferred biometric data or information, it should be understood that some other forms of biometrics may be used and accordingly some other biometric device may be used to substitute the fingerprint scanner/reader 106a.

The fingerprint reader 106a of the fingerprint information transfer station 106 of FIG. 1 reads the fingerprint information/data obtained from the patient 202 and records/stores the same on a server 108 for future reference or patient recognition purposes. The server 108 in the context of the present invention is a computer, PC, or any other electronic computing device having a processor, memory, etc usually used for the storage and processing of informational data. The server 108 in some other embodiment may be a cloud server. Apart from storing the fingerprint information/data on the server 108, the fingerprint scanner/reader 106a upon reading the fingerprint information from patient 202 electronically transfers the recorded patient's fingerprint information to a lid 102 of a pill container 101 usually kept/placed on the fingerprint information transfer station 106. In some other embodiment, the fingerprint information from the patient 202 is electronically transferred to the fingerprint information transfer station 106 instead of its lid 102. The electronic transfer of the fingerprint information recorded by the scanner 106a is a digital copy, electronic authorization, or any other technique known in the art. In operation, the pharmacist 201 prefills the pill dispensing container/dispenser 101 with the prescribed pills, and before delivery to the patient 202 places the lid 102 of the pill container 101 or the pill container 101 in the fingerprint information transfer station 106 to ensure the patient 202 scans his/her fingerprints on the reader 106a and the read fingerprint information gets transferred and linked to the pill container/dispenser 101. According to an embodiment, if the patient 202 is determined to be a new patient by the server 108 or pharmacy records then the patient 202 is required to scan the fingerprints on the reader 106a to transfer and link the patient's fingerprint information to the pill container 101 prefilled with the pills the patient 202 desires to purchase from the pharmacy. Likewise, if the patient 202 is determined to be a recurring patient who had in the past purchased the same prescribed pills or other pills then the existing fingerprint data/information is automatically linked to the pill container 101 without requiring the patient 202 to scan his/her fingerprints again using the reader 106a. The stored information on the server 108 is used in this condition and electronically transferred to the pill container 101. In some other embodiment, irrespective of if the patient 202 is an existing purchaser of the medication or not, the patient 202 is required to scan his/her fingerprints using the scanner 106a of the fingerprint information transfer station 106 to link his/her fingerprint information to the pill container 101 prefilled with the pills that the patient 202 wishes to purchase from the pharmacy 200. Once it is ensured that the fingerprint information is linked to the prefilled pill container 101 by the pharmacist, the pill container 101 is then delivered to the patient 202.

Referring to FIG. 2, the fingerprint information transfer station 106 is shown embodied in the form of a handheld device. As seen, in this embodiment, the fingerprint information transfer station 106 is configured as a portable fingerprint transfer device 107 comprising essentially of a body 107a, a port 107b to be connected to the pill container 101 (particularly the lid 102), and electronic circuitry (not seen) housed inside the body 107a. The device 107 also embodies a fingerprint scanner/reader 107c. This form of fingerprint transfer device 107 is more convenient for use and is portable for a pharmacist 201 to carry and move around. The device 107 of FIG. 2 is shown connected to the lid 102 of the pill container 101 as seen in FIG. 3 as will be described in detail in the description to follow. In operation, the portable fingerprint transfer device 107 is capable of recording or linking the fingerprint information associated with the patient 202 to the pill container 101 similar to the fingerprint information transfer station 106 described with respect to FIG. 1.

As a very important aspect of the present invention, the patient 104 once have an access to the pill container/dispenser 101 from pharmacy 200, the patient 202 can dispense the pills out of the container 101 as and when he/she wants by authenticating himself/herself using a fingerprint scanner/reader 102a configured on the pill container 101 or by authenticating and then manually pressing a button (not shown) provided on the pill container 101, which will be described in detail in the description to follow.

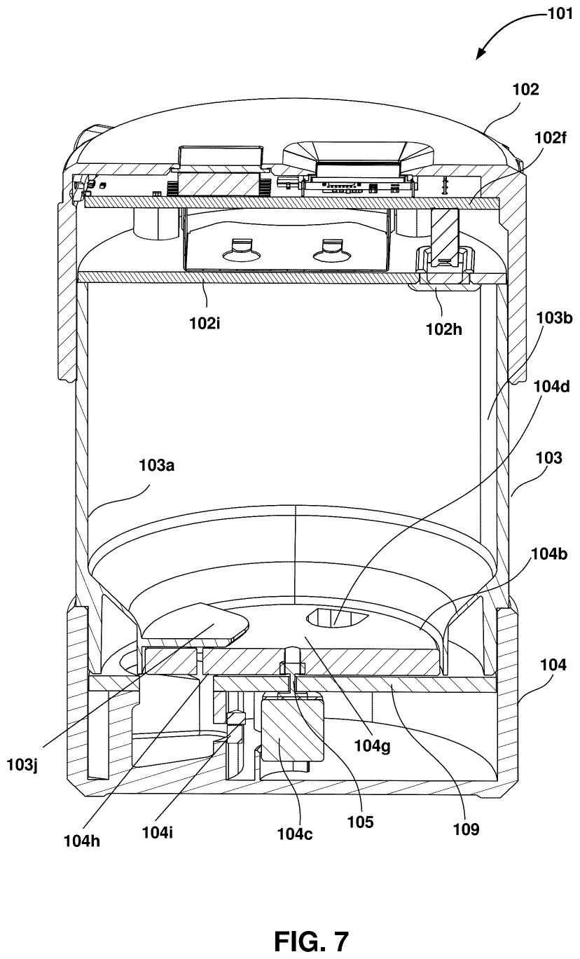

Referring to FIGS. 4-9, the pill container 101 comprises a lid 102. The lid 102 may include a fingerprint scanner/reader 102a to allow the patient/user 202 to scan his fingerprints in order to dispense the stored pills out of the container 101. In the context of the present invention, it is important that a biometric device be integrated into the pill container 101 as in the fingerprint information transfer station 106 to read the fingerprint information of the patient 202. The preferred embodiment shows the configuration of the scanner/reader 102a on the lid 102. It should be understood that the fingerprint scanner/reader 1102a may be configured in any other place on the pill container 101. In operation, upon scanning the fingerprint using the scanner/reader 102a, the patient 202 is recognized and gets authenticated and then the dispensing mechanism housed within the pill container 101 gets activated to dispense the pills for consumption by the patient 202. In some other embodiment, the patient 202 may need to press a button (not shown) located on the pill container 101 after fingerprint authentication to activate dispensing mechanism. The lid 102 further houses a circuitry 102f. The circuitry 102f includes necessary electronic components such as controller/processors, battery, memory, and software routines programmatically stored in the memory for the operation of the pill container 101. The circuitry 102f is configured to at least facilitate reading fingerprints of the patient 202 by the reader 102a to authenticate the patient 202 to initiate the dispensing mechanism for the pills and drive a rotary motor 104c (FIG. 7) (E.g., a stepper motor) configured within the pill container/dispenser 101 to dispense the pills.

The lid 102 of the pill container 101 further includes a display screen 102b configured to display information related to pills stored in the pill container 101. For example, but not limited to, information on how many pills are remaining in the pill container 101 or the count of the pills. The display may be an LED screen or an LCD screen known in the art. The lid 102 may also include one or more indicators 102c to indicate to the patient, how many times a day the patient is supposed to consume the pills. The lid 102 may further include a port 102k that may be selectively used to make programming changes to the circuitry 102f housed in the lid 102 or update the program. The lid 102 may further include ridges 102d extending from the top end to the bottom end on the exterior surface. The ridges 102d provide strength to the lid 102. The underside of the lid 102 comprises a set of grooves/slots 102c.

The pill container 101 further includes a base 104. The base 104 includes a cavity 104f, and an opening 104a. The cavity 104f of the base 104 houses several components and structural designs such as a rotating disc 104b, a motor 104c operationally connected to the rotating disc 104b, an optic sensor 104i, a circuitry (not seen), and so on. The opening 104a is responsible for dispensing out the pills. The rotating disc 104b is disposed of within the cavity 104f of the base 104. As seen, the disc 104b comprises a top surface 104g, a bottom surface 104h, and at least one dispensing slot 104d.

Although in the preferred example, the disc 104b is shown to include four slots 104d, it should be understood that the disc 104b may include more or a smaller number of such slots. The slots 104d, as seen, may vary in dimension/size, and only one of the required slot 104d is made operational. This means at a given time only one slot is opened (while others are made to remain closed) and allowed to dispense the pills. The opened slot 104d on disc 104b is further covered by a flap 103j configured within a sleeve portion 103. The selection of the slot 104d (for opening) depends upon the size of the pills being filled within the pill container 101 by the pharmacist 201. Depending upon the pills filled up in the pill container 101, the required slot 104d out of all the available slots (on the disc 104b) is made operational. In an example scenario, out of 4 slots 104d, the three slots are made to remain scaled and one of the slots is to remain unsealed for dispensing the pills out of the container 101. The rotating disc 104b is provided with a provision 104j at its center to receive an end of a shaft 105 of the rotary motor 104c. The motor 104c is configured within the pill container 101 such that the motor 104c is electrically connected to the circuitry 102f housed within the lid 102. The base 104 further includes a set of engaging protrusions 104e (formed within an interior wall of the base 104) that engage to a set of grooves 103d externally formed at the bottom of the sleeve 103. The disc 104b (the bottom surface 104h) is further supported by a rigid structure 109 formed within the base 104 as seen in FIG. 7.

The sleeve 103 of the pill container 101 includes a sidewall 103a with a top opening 103c and a bottom opening 103f. The sleeve 103 comprises a set of engaging protrusion 103c formed at the top of the sleeve 103. The sleeve 103 further comprises a casing 103b through which a data and communication cable 102g is routed to connect the circuitry 102f housed within the lid 102 with a circuitry (not seen) housed within the base 104. The casing 103b is attached to the interior of the sidewall 103a of the sleeve 103. The data and communication cable 102g transfers the data and communication from the base 104 to the lid 102 and vice versa. For example, once the optic sensor 104i detects the pills are dispensed, the information is passed onto the circuitry of the lid 102 (via data and communication cable 102g) so that the count of the pills within the pill container 101 can be reduced.

The sleeve 103 is assembled with the base 103 and the lid 102 to form the pill container 101. The sleeve 103 engages to the lid 102 using the set of engaging protrusion 103c and the grooves 102e located on the lid 102. The sleeve 103 engages to the base 104 using the set of engaging protrusion 104e located on the base 104 and the grooves 103d located on the sleeve 103. The locking mechanism shown is crucial and essential as it safeguards access to the pills contained inside the pill container from the children, thereby preventing and misuse of pills by children. In another embodiment, the lid 102 and the base 104 may be rotatably assembled with the sleeve 103 or using some other mechanism known in the art. It might also be possible to form the sleeve 103 and base 104 as an integrated single unitary product. The lid 102, as seen, acts as a closure for the top opening 103e of the sleeve 103, and the base 104 acts as a closure for the bottom opening 103f of the sleeve 103. With the lid 102 and base 104 assembled with the sleeve 103, the so-assembled configuration serves to store the prescribed pills within the interior cavity within the sleeve 103.

In operation, upon having the pill container 101 in possession, the patient 202 has to authenticate himself by scanning his/her fingerprints using the fingerprint scanner 102a located on the lid 102 of the pill container 101. Upon successful authentication, the circuitry 102f will trigger the dispensing mechanism to dispense the pills out of the pill container 101. The dispensing mechanism in general in the context of the present invention refers to the operation of the rotary motor 104c. When the motor 104c is operational, it rotates the shaft 105 of the motor 104c which in turn rotates the rotating disc 104b to one full round of rotation ensuring the dispensing slot 104d of the disc 104b that is open gets aligned or oriented with the opening 104a of the base 104 to dispense pill out of the pill container 101. The at least one dispensing slot (104d) that's open and oriented over the opening (104a) of the base (104) is sized according to the pills contained inside the sleeve (103) to dispense the pills upon activation of the dispensing mechanism. If one pill needs to be dispensed the disc 104b is programmed to rotate once, if two pills are to be dispensed under a dose then the disc 104b will be programmed to rotate twice, and so on. During this operation of dispensing the pills, the flap 103j of the sleeve 103 ensures no more pills get passed down from the sleeve 103 to the base 104 and dispensed out through the opening 104a creating a barrier.

Referring to FIGS. 2-3, and 7, the circuitry 102f is housed within the lid 102 and is concealed using a separator 102i from the sleeve 103. The separator 102i also includes an openable cap 102h that when opened reveals a connecting port (not shown) to which the handheld device 107 connects for transferring the fingerprint information when the patient purchases the prescribed pills from the pharmacy 200. During the normal course of use of the pill container 101, the cap 102h remains in place and does not allow any pills from the sleeve 103 to get into the lid 102. Whenever the fingerprint information needs to be recorded or linked to pill container 101 then the cap 102h is opened and the port 107b of the portable fingerprint transfer device 107 is connected to the port disposed just inside the cap 102h.

Optionally, in the embodiment described above, it may be possible for a pharmacist 201 at the pharmacy 200 and the patient 202 to have a computer software program product installed in their communication devices (such as a smart phone) to get an update related to pill dispenser and/or health of the patient. The alerts may be in the form of reminders to take pills to the patient or in the form of an update on the number of pills remaining in the pill container that may be useful to both the patient 202 and the pharmacy 200.

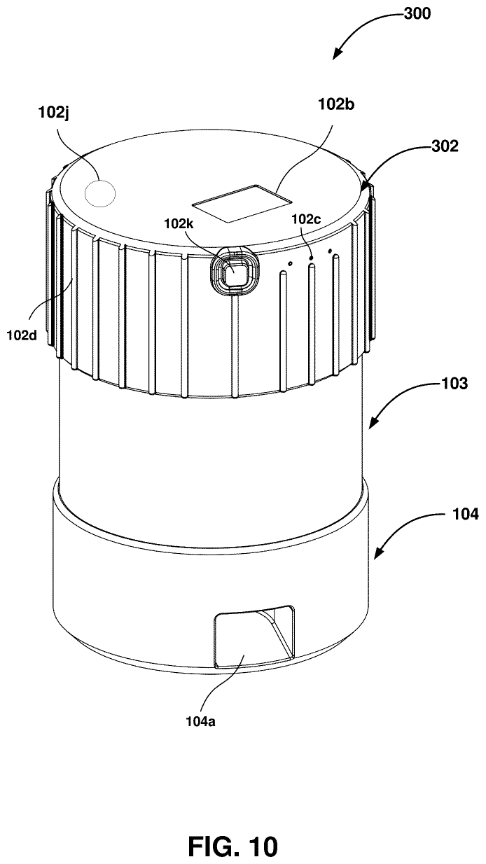

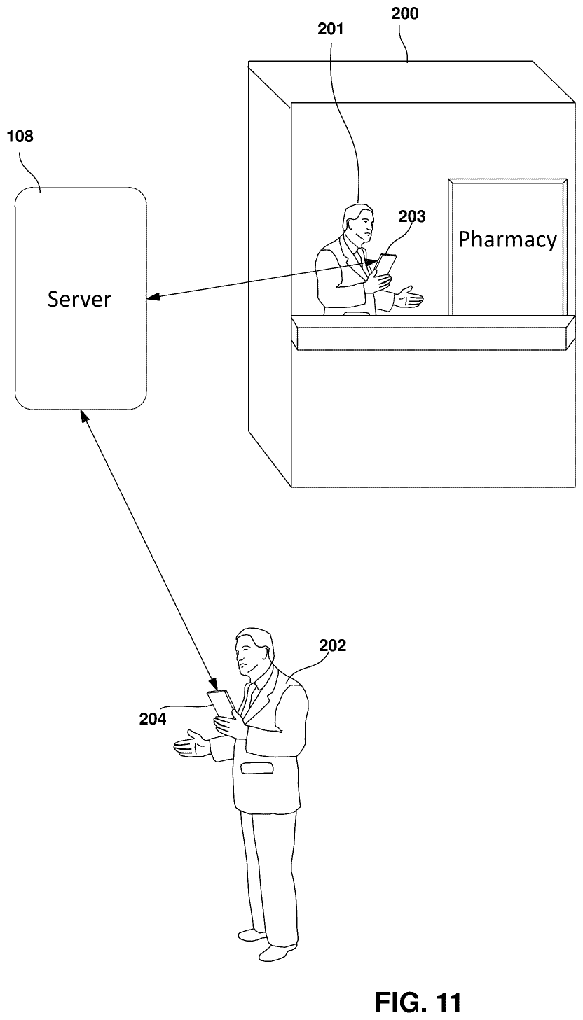

Referring to FIGS. 10 and 11 that show a pill container and a pill dispensing system and method according to another embodiment of the present invention. In this embodiment, the pill container 300 is essentially identical to the pill container 101 described above except the pill container 300 does not include a fingerprint reader 102a configured on the lid 102. The other technicalities with respect to the lid 102, the sleeve 103, and the base 104 and associated components remain identical as described above with respect to FIGS. 4-9.

The pill container 300 shown in this embodiment is essentially capable of working without recording any biometric information. In this embodiment, essentially a software program product or an app is installed over a patient's communication device 204, and the same program product is installed over a pharmacist's communication device 203. The communication devices 203 and 204 of the pharmacist 201 and the patient 202 respectively are capable of communicating with a server 108 over a network (such as the internet or other known communication networks). In this embodiment, the patient 202 does not need to go through recording and linking any biometric information and then authenticating the same for dispensing the pills. Rather, in this embodiment, the patient 202 just needs to install the app or software program product on the patient device 204 to receive alerts on the device 204. The alerts may be related to the timely intake of pills notifying the patients to take their pills, notifying about the doses of the pills the patient needs to take, and so on. Likewise, the computer program product installed on the pharmacist device 201 also alters the pharmacy about the pills remaining in the pill dispensing container, about health improvement of the patient, and so on. The server 108 may be a cloud server capable of storing and transmitting information to the pharmacy 201 and the patient 202. The communication devices 203,204 associated with the pharmacist 201 and the patient 202 may include but are not limited to smartphones, laptops, computers, and so on. The devices 203,204 include all processing, storage, and display components, voice components, and so on. As seen in FIG. 11, in this embodiment, essentially the pharmacist 201 and the patient 202 get updates about the medication pills, the patient's health, and so on. In this embodiment, the lid 102 of the pill container 303 may include a button 102j to activate the dispensing mechanism housed inside the pill container 303 according to an embodiment. In another embodiment, the app installed in the patient related device 204 may itself allow the patient 202 to activate the pill dispensing mechanism of the pill container 300.

The different parts/components forming the pills container/dispenser 101,300 and the fingerprint transfer station/device 106, 107 may be made in different shapes and sizes using different materials and thus any variation or substitution of the components is very much possible by persons skilled in the art.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

What is claimed is:

1. A pill dispensing system (100), comprising:
a pill container (101) having
a base (104) with a cavity (104f) for housing at least a rotating disc (104b), a rotary motor (104c) operationally connected to the rotating disc (104b), and an opening (104a) for dispensing a pill out of the pill container (101);
a lid (102) embodying at least a fingerprint reader (102a), a circuitry (102f) configured to at least read the fingerprint information of a patient (202), and drive a rotary motor (104c);
a sleeve (103) with sidewalls (103a), a top opening (103e), and a bottom opening (103f), wherein the sleeve (103) is configured to receive the lid (102) as a closure for the top opening (103e), and the base (104) as a closure for the bottom opening (103f), wherein with the lid (102) and the base (104) closed, the sleeve (208) retains pills therein;
wherein the rotating disc (104b) comprising a top surface (104g), a bottom surface (104h), and at least one dispensing slot (104d), wherein the rotating disc (104b) is operationally coupled to the rotary motor (104c) to rotate and complete a full round of rotation and orient the at least one dispensing slot (104d) over the opening (104a) of the base (104) to dispense the pills upon activation of the dispensing mechanism; and
a fingerprint information transfer medium (106,107) equipped with a fingerprint scanner/reader (106a,107c), wherein the fingerprint transfer medium (106,107) records the patient's fingerprint and electronically transfers the recorded patient's fingerprint information to the pill container (101) for linking the patient's fingerprint to the pill container (101) to allow the patient (104) to authenticate and dispense the pills out of the pill container (101).

2. The pill dispensing system (100) of claim 1, wherein upon reading the fingerprint of a patient (202), the circuitry (102f) activates the dispensing mechanism.

3. The pill dispensing system (100) of claim 2, wherein the dispensing mechanism involves activation of a rotary motor (104c) which in turn rotates the rotating disc (104b) to complete a full round of rotation and orient a dispensing slot (104d) over the opening (104a) of the base (104) to dispense the pills out of the pill container (101).

4. The pill dispensing system (100) of claim 1, wherein the base (104) comprises a first set of engaging protrusions (104e) configured within an interior wall of the base (104).

5. The pill dispensing system (100) of claim 1, wherein the sleeve (103) comprises a second set of engaging protrusions (103c) formed at the top of the sleeve (103) and a first set of grooves (103d) formed at the bottom of the sleeve (103).

6. The pill dispensary system (100) of claim 1, wherein the lid (102) comprises a second set of grooves (102e) configured within an interior wall of the lid (102).

7. The pill dispensary system (100) of claim 4, wherein the first set of engaging protrusions (104e) of the base (104) engages with the first set of grooves (103d) of the sleeve (103) to connect the base (104) with the sleeve (103).

8. The pill dispensary system (100) of claim 5, wherein the second set of engaging protrusions (103c) of the sleeve (103) engages with the second set of grooves (102e) of the lid (102) to connect the lid (102) with the sleeve (103).

9. The pill dispensing system (100) of claim 1, wherein the rotating disc (104b) is supported by a rigid structure (109) configured within the base (104).

10. The pill dispensing system (100) of claim 1, wherein the base (104) further comprises at least one optic sensor (104i) to detect the pills dispensed out of the pill container (101) and communicate the information to the circuitry (102f) of the lid (102) via a data and communication cable (102g) disposed within the sleeve (103).

11. The pill dispensing system (100) of claim 1, wherein the lid (102) further comprising
a display screen (102b) configured to display information related to pills stored in the pill container (101); and
a port (102k) selectively usable to make programming changes to the circuitry (102f) housed in the lid (102).

12. The pill dispensing system (100) of claim 1, wherein the fingerprint information transfer medium (106,107) is at least
a fingerprint information transfer station (106) that's stationary and configured at a pharmacy (200) at an appropriate location; and,
a portable fingerprint information transfer device (107) is a handheld device carriable by a pharmacist.

13. The pill dispensing system (100) of claim 1, wherein the lid (102) further comprises a separator (102i) that conceals the circuitry (102f) from the sleeve (103) and wherein the separator (102i) further has an openable cap (102h) that reveals a connecting port to which the portable fingerprint information transfer device (107) is connected for transferring the fingerprint information when the patient purchases the prescribed pills from the pharmacy (200.)

14. The pill dispensing system (100) of claim 10, wherein the data and communication cable (102g) is routed through a casing (103b) attached to the interior of the sidewall 103a of the sleeve (103).

15. The pill dispensing system (100) of claim 1, wherein the at least one dispensing slot (104d) oriented over the opening (104a) of the base (104) is sized according to the pills contained inside the sleeve (103) and is open to dispense the pills upon activation of the dispensing mechanism.

16. The pill dispensing system (100) of claim 15, wherein the at least one opened slot (104d) on the rotating disc (104b) is covered by a flap (103j) configured within a sleeve portion (103) to restrict unwanted pills from getting dispensed.

17. The pill dispensing system (100) of claim 1 further comprising a computer software program product installed over a first communication device (203) associated with a pharmacist (201) and a second communication device (204) associated with a patient (202).

18. The pill dispensing system (100) of claim 17, wherein the computer software program product when executed by the first communication device (203) and the second communication device (204) generates at least an alert related to the health of the patient (202), alerts to the patient on taking pills, alert on the number of pills remaining in the pill container (101) useful to both the patient (202) and the pharmacy (200).

19. The pill dispensing system (100) of claim 1 further comprising a server (108) for storing fingerprint information/data obtained from the patient (202) for future reference or patient recognition purposes.

20. A pill dispensing system (100), comprising:
a pill container (101) having
a base (104) with a cavity (104f) for housing at least a rotating disc (104b), a rotary motor (104c) operationally connected to the rotating disc (104b), and an opening (104a) for dispensing a pill out of the pill container (101);
a lid (102) embodying at least a circuitry (102f) configured to at least read the fingerprint information of a patient (202), and drive a rotary motor (104c);
a sleeve (103) with sidewalls (103a), a top opening (103e), and a bottom opening (103f), wherein the sleeve (103) is configured to receive the lid (102) as a closure for the top opening (103e), and the base (104) as a closure for the bottom opening (103f), wherein with the lid (102) and the base (104) closed, the sleeve (208) retains pills therein;
wherein the rotating disc (104b) comprising a top surface (104g), a bottom surface (104h), and at least one dispensing slot (104d), wherein the rotating disc (104b) is operationally coupled to the rotary motor (104c) to rotate and complete a full round of rotation and orient the at least one dispensing slot (104d) over the opening (104a) of the base (104) to dispense the pills upon activation of the dispensing mechanism; and
a computer software program product installed on a first communication device (203) associated with a pharmacist (201) and a second communication device (204) associated with a patient (202), wherein the computer software program product when executed by the first communication device (203) and the second communication device (204) generates at least an alert related to the health of the patient (202), alerts to the patient on taking pills, alert on the number of pills remaining in the pill container (101) useful to both the patient (202) and the pharmacy (200).
